SEQUENCE LISTING (14 sequences)

SEQ ID NO: 1 - DNA, 51 bp, Artificial
  Encodes a His rich linker sequence
ctagtgccgcgcggcagccaccaccaccaccaccaccaccaccaccactag

SEQ ID NO: 2 - protein, 16 aa, Artificial
  His rich linker sequence cleavable by thrombin protease
LVPRGSHHHHHHHHHH

SEQ ID NO: 3 - DNA, 30 bp, Artificial
  Encodes a His chain for labelling receptors in their intracellular loop
agatcccatcatcaccatcaccacggatct

SEQ ID NO: 4 - protein, 10 aa, Artificial
  His rich chain for labelling receptors in their intracellular loop
RSHHHHHHGS

SEQ ID NO: 5 - DNA, 21 bp, Artificial
  Encodes a His chain for labeling receptor G alpha fusion proteins
catcatcaccatcaccactaa

SEQ ID NO: 6 - protein, 6 aa, Artificial
  His chain for labelling receptor G alpha fusion protein
HHHHHH

SEQ ID NO: 7 - DNA, 30 bp, Artificial
  Encodes a linking sequence to fuse rat serotonin receptor 5HT2C and human alpha subunit G-alpha q
ggatccggaggtggatggggaggtggatcc

SEQ ID NO: 8 - protein, 10 aa, Artificial
  Linking sequence encoded by SEQ ID NO 7
GSGGGWGGGS

SEQ ID NO: 9 - protein, 413 aa, Homo sapiens
MGQPGNGSAFLLAPNRSHAPDHDVTQQRDEVWVVGMGIVMSLIVLAIVFGNVLVITAIAKFERLQTVTNYFITSLACADLVMGLAVVPFGAAHILMKMWTFGNFWCEFWTSIDVLCVTASIETLCVIAVDRYFAITSPFKYQSLLTKNKARVIILMVWIVSGLTSFLPIQMHWYRATHQEAINCYANETCCDFFTNQAYAIASSIVSFYVPLVIMVFVYSRVFQEAKRQLQKIDKSEERFHVQNLSQVEQDGRTGHGLRRSSKFCLKEHKALKTLGIIMGTFTLCWLPFFIVNIVHVIQDNLIRKEVYILLNWIGYVNSGFNPLIYCRSPDFRIAFQELLCLRRSSLKAYGNGYSSNGNTGEQSGYHVEQEKENKLLCEDLPGTEDFVGHQGTVPSDNIDSQGRNCSTNDSLL

SEQ ID NO: 10 - DNA, 2015 bp, Homo sapiens
actgcgaagcggcttcttcagagcacgggctggaactggcaggcaccgcgagcccctagcacccgacaagctgagtgtgcaggacgagtccccaccacacccacaccacagccgctgaatgaggcttccaggcgtccgctcgcggcccgcagagccccgccgtgggtccgcccgctgaggcgcccccagccagtgcgcttacctgccagactgcgcgccatggggcaacccgggaacggcagcgccttcttgctggcacccaatagaagccatgcgccggaccacgacgtcacgcagcaaagggacgaggtgtgggtggtgggcatgggcatcgtcatgtctctcatcgtcctggccatcgtgtttggcaatgtgctggtcatcacagccattgccaagttcgagcgtctgcagacggtcaccaactacttcatcacttcactggcctgtgctgatctggtcatgggcctggcagtggtgccctttggggccgcccatattcttatgaaaatgtggacttttggcaacttctggtgcgagttttggacttccattgatgtgctgtgcgtcacggccagcattgagaccctgtgcgtgatcgcagtggatcgctactttgccattacttcacctttcaagtaccagagcctgctgaccaagaataaggcccgggtgatcattctgatggtgtggattgtgtcaggccttacctccttcttgcccattcagatgcactggtaccgggccacccaccaggaagccatcaactgctatgccaatgagacctgctgtgacttcttcacgaaccaagcctatgccattgcctcttccatcgtgtccttctacgttcccctggtgatcatggtcttcgtctactccagggtctttcaggaggccaaaaggcagctccagaagattgacaaatctgagggccgcttccatgtccagaaccttagccaggtggagcaggatgggcggacggggcatggactccgcagatcttccaagttctgcttgaaggagcacaaagccctcaagacgttaggcatcatcatgggcactttcaccctctgctggctgcccttcttcatcgttaacattgtgcatgtgatccaggataacctcatccgtaaggaagtttacatcctcctaaattggataggctatgtcaattctggtttcaatccccttatctactgccggagcccagatttcaggattgccttccaggagcttctgtgcctgcgcaggtcttctttgaaggcctatgggaatggctactccagcaacggcaacacaggggagcagagtggatatcacgtggaacaggagaaagaaaataaactgctgtgtgaagacctcccaggcacggaagactttgtgggccatcaaggtactgtgcctagcgataacattgattcacaagggaggaattgtagtacaaatgactcactgctgtaaagcagtttttctacttttaaagacccccccccccccaacagaacactaaacagactatttaacttgagggtaataaacttagaataaaattgtaaaaattgtatagagatatgcagaaggaagggcatccttctgccttttttatttttttaagctgtaaaaagagagaaaacttatttgagtgattatttgttatttgtacagttcagttcctctttgcatggaatttgtaagtttatgtctaaagagctttagtcctagaggacctgagtctgctatattttcatgacttttccatgtatctacctcactattcaagtattaggggtaatatattgctgctggtaatttgtatctgaaggagattttccttcctacacccttggacttgaggattttgagtatctcggacctttcagctgtgaacatggactcttcccccactcctcttatttgctcacacggggtattttaggcagggatttgaggagcagcttcagttgttttcccgagcaaaggtctaaagtttacagtaaataaaatgtttgaccatg

SEQ ID NO: 11 - protein, 460 aa, Rattus norvegicus
MVNLGNAVRSLLMHLIGLLVWQFDISISPVAAIVTDTFNSSDGGRLFQFPDGVQNWPALSIVVIIIMTIGGNILVIMAVSMEKKLHNATNYFLMSLAIADMLVGLLVMPLSLLAILYDYVWPLPRYLCPVWISLDVLFSTASIMHLCAISLDRYVAIRNPIEHSRFNSRTKAIMKIAIVWAISIGVSVPIPVIGLRDESKVFVNNTTCVLNDPNFVLIGSFVAFFIPLTIMVITYFLTIYVLRRQTLMLLRGHTEEELANMSLNFLNCCCKKNGGEEENAPNPNPDQKPRRKKKEKRPRGTMQAINNEKKASKVLGIVFFVFLIMWCPFFITNILSVLCGKACNQKLMEKLLNVFVWIGYVCSGINPLVYTLFNKIYRRAFSKYLRCDYKPDKKPPVRQIPRVAATALSGRELNVNIYRHTNERVARKANDPEPGIEMQVENLELPVNPSNVVSERISSV

SEQ ID NO: 12 - DNA, 1383 bp, Rattus norvegicus
atggtgaaccttggcaacgcggtgcgctcgctcctgatgcacctaatcggcctattggtttggcaattcgatatttccataagtccagtagcagctatagtaactgacacttttaattcctccgatggtggacgcttgtttcaattcccggacggggtacaaaactggccagcactttcaatcgtcgtgattataatcatgacaatagggggcaacattcttgttatcatggcagtaagcatggagaagaaactgcacaatgcaaccaattacttcttaatgtccctagccattgctgatatgctggtgggactacttgtcatgcccctgtccctgcttgctattctttatgattatgtctggcctttacctagatatttgtgccccgtctggatttcactagatgtgctattttcaactgcgtccatcatgcacctctgcgccatatcgctggaccggtatgtagcaatacgtaatcctattgagcatagccggttcaattcgcggactaaggccatcatgaagattgccatcgtttgggcaatatcaataggagtttcagttcctatccctgtgattggactgagggacgaaagcaaagtgttcgtgaataacaccacgtgcgtgctcaatgaccccaacttcgttctcatcgggtccttcgtggcattcttcatcccgttgacgattatggtgatcacctacttcttaacgatctacgtcctgcgccgtcaaactctgatgttacttcgaggtcacaccgaggaggaactggctaatatgagcctgaactttctgaactgctgctgcaagaagaatggtggtgaggaagagaacgctccgaaccctaatccagatcagaaaccacgtcgaaagaagaaagaaaagcgtcccagaggcaccatgcaagctatcaacaacgaaaagaaagcttccaaagtccttggcattgtattctttgtgtttctgatcatgtggtgcccgtttttcatcaccaatatcctgtcggttctttgtgggaaggcctgtaaccaaaagctaatggagaagcttctcaatgtgtttgtgtggattggctatgtgtgttcaggcatcaatcctctggtgtacactctctttaataaaatttaccgaagggctttctctaaatatttgcgctgcgattataagccagacaaaaagcctcctgttcgacagattcctagggttgctgccactgctttgtctgggagggagctcaatgttaacatttatcggcataccaatgaacgtgtggctaggaaagctaatgaccctgagcctggcatagagatgcaggtggagaacttagagctgccagtcaacccctctaatgtggtcagcgagaggattagtagtgtgtaa

SEQ ID NO: 13 - protein, 422 aa, Homo sapiens
MDVLSPGQGNNTTSPPAPFETGGNTTGISDVTVSYQVITSLLLGTLIFCAVLGNACVVAAIALERSLQNVANYLIGSLAVTDLMVSVLVLPMAALYQVLNKWTLGQVTCDLFIALDVLCCTSSILHLCAIALDRYWAITDPIDYVNKRTPRRAAALISLTWLIGFLISIPPMLGWRTPEDRSDPDACTISKDHGYTIYSTFGAFYIPLLLMLVLYGRIFRAARFRIRKTVKKVEKTGADTRHGASPAPQPKKSVNGESGSRNWRLGVESKAGGALCANGAVRQGDDGAALEVIEVHRVGNSKEHLPLPSEAGPTPCAPASFERKNERNAEAKRKMALARERKTVKTLGIIMGTFILCWLPFFIVALVLPFCESSCHMPTLLGAIINWLGYSNSLLNPVIYAYFNKDFQNAFKKIIKCKFCRQ

SEQ ID NO: 14 - DNA, 1308 bp, Homo sapiens
ccttcccgcgccgggcgcgcaggcatggatgtgctcagccctggtcagggcaacaacaccacatcaccaccggctccctttgagaccggcggcaacactactggtatctccgacgtgaccgtcagctaccaagtgatcacctctctgctgctgggcacgctcatcttctgcgcggtgctgggcaatgcgtgcgtggtggctgccatcgccttggagcgctccctgcagaacgtggccaattatcttattggctctttggcggtcaccgacctcatggtgtcggtgttggtgctgcccatggccgcgctgtatcaggtgctcaacaagtggacactgggccaggtaacctgcgacctgttcatcgccctcgacgtgctgtgctgcacctcatccatcttgcacctgtgcgccatcgcgctggacaggtactgggccatcacggaccccatcgactacgtgaacaagaggacgccccggcgcgccgctgcgctcatctcgctcacttggcttattggcttcctcatctctatcccgcccatgctgggctggcgcaccccggaagaccgctcggaccccgacgcatgcaccattagcaaggatcatggctacactatctattccacctttggagctttctacatcccgctgctgctcatgctggttctctatgggcgcatattccgagctgcgcgcttccgcatccgcaagacggtcaaaaaggtggagaagaccggagcggacacccgccatggagcatctcccgccccgcagcccaagaagagtgtgaatggagagtcggggagcaggaactggaggctgggcgtggagagcaaggctgggggtgctctgtgcgccaatggcgcggtgaggcaaggtgacgatggcgccgccctggaggtgatcgaggtgcaccgagtgggcaactccaaagagcacttgcctctgcccagcgaggctggtcctaccccttgtgcccccgcctctttcgagaggaaaaatgagcgcaacgccgaggcgaagcgcaagatggccctggcccgagagaggaagacagtgaagacgctgggcatcatcatgggcaccttcatcctctgctggctgcccttcttcatcgtggctcttgttctgcccttctgcgagagcagctgccacatgcccaccctgttgggcgccataatcaattggctgggctactccaactctctgcttaaccccgtcatttacgcatacttcaacaaggactttcaaaacgcgtttaagaagatcattaagtgtaagttctgccgccagtgatgacggaggagtagc

(19) United States
(12) Patent Application Publication
(10) Pub. No.: US 2008/0032418 A1
(43) Pub. Date: Feb. 7, 2008

(54) Cell Free Assay Systems For Identifying A Substance Of Interest

(76) Inventors: David Adams, New York, NY (US); Kevin J. Lee, New York, NY (US)

Correspondence Address: INVITROGEN CORPORATION;C/O INTELLEVATE, P.O. BOX 52050, MINNEAPOLIS, MN, 55402, US

(21) Appl. No.: 10/543,674

(22) PCT Filed: Feb. 9, 2004
(86) PCT No.: PCT/US04/03906

§ 371 Date: Sep. 19, 2007

Related U.S. Application Data
(60) Provisional application No. 60/446,242, filed on Feb. 10, 2003.

(51) Int. Cl. G01N 33/53; C07K 14/705

(52) U.S. Cl. 436/501; 530/402

(57) ABSTRACT

The invention relates to an assay for determining if ligands bind to a receptor. The method involves the use of complexes, where the receptor is linked to a reporter molecule via an (His)ₙ chain. When ligand binds to the receptor, the reporter molecule, which is preferably a peryline, yields a signal.

### FIELD OF THE INVENTION

This invention relates to cell free assays for determining if a substance of interest interacts with a molecule of interest, such as receptor protein. It also relates to the constructs which are useful in these cell free assays.

### BACKGROUND AND PRIOR ART

Cell based screening assays are tools well known to biologists. In these assays one investigates compounds of interest to determine, e.g. if the compounds modulate one or more biological process of interest.

One area where cell based screening assays have become widely accepted is the high-throughput analysis of materials for use as pharmaceuticals. In these assays the modulation of a given target by a compound of interest is coupled to a specific cellular readout. For example, the activation of a given cell surface receptor can elicit a change in the transcriptional profile of an enzymatic reporter gene, or the elevation of a given second messenger such as Ca²⁺, cAMP, or inositol triphosphate, which can be quantified with a fluorescent or calorimetric measurement. Cell-based assays are useful and desirable because, unlike traditional binding assays, they have the potential to measure receptor modulating activity, a feature that is, ultimately, a requirement of drug functions. Cell-based screening assays also have several advantageous over animal model testing (e.g., lower expense, shorter assay period). High-throughput, cell-based screening assays can be scaled up via technologies such as “FLIPR,” “Leedseeker,” “VIPR,” and fluorescent, high speed cell-imaging.

However, carrying out high-throughput, cell-based assays presents a set of distinct challenges. Unlike biochemical reagents like enzymes, proteins, and membrane-bound receptors, cells are live, dynamic entities. In some cases the complex biological processes of a cell can negatively impact the proper expression and function of a recombinant target molecule in a heterologous cell system. There are numerous examples of target molecules (e.g., olfactory receptors) that can not be expressed in cell lines that are amenable to screening of target proteins that have a detrimental effect on the viability of the host cell when expressed at high levels. Cell-based assays are dependent upon the biological responsiveness of the cells in an appropriate assay platform, and the proper and consistent expression of the target molecule. Constructing a robust cell-based assay can be problematic for targets that have a negative effect on cell viability over time as cells expressing the target molecule are subcultured. A further complication is the requirement to couple the activity of the target to a measurable cellular process, which can be very difficult in the case of uncharacterized targets (e.g., orphan receptors). Further, the miniaturization of cell-based screening assays is progressing, with smaller and smaller numbers of cells being used. As this occurs, sensitivity of the assay to variability increases rapidly and dramatically.

Due to the issues with cell-based screening assays, some but not all of which are discussed herein, there has been, and continues to be interest in cell free screening assays. Schmid, et al, Anal. Chem 70:1331-1338 (1998), for example, discuss chip-based screening assays, as do Hovius, et al, Trends Pharmacol. Sci. 21:266-273 (2000), and Weiss, Nat. Struct. Biol. 7:724-729 (2000). These systems can permit single molecule resolution studies, as are described by, e.g., Nie, et al, Annu. Rev. Biophys. Biomol. Struct. 26:567-596 (1997); Ambrose, et al, Chem. Rev. 99:2929-2956 (1999), Weiss, Science 283:1676-1683 (1999).

Besides numerous advantages in terms of lower set-up and reagent costs, and the ability to assay uncharacterized receptors, such chip-based assays offer the potential to assay multiple targets simultaneously. Multiplex, chip-based assays have the potential to profile the activity of a given compound on a battery of receptors to evaluate, for example, the selectivity of a given compound. These assays also could be used to identify a previously unknown receptor for a given ligand.

Such chip-based assays are not without problems, however. Basic to such assays is immobilization of the target molecule, such as a receptor protein, to the solid phase. Interactions between the solid phase and the immobilized molecule may lead to modification or loss of function of the immobilized molecule. Yet another issue which must be confronted is that these chip-based systems may not fairly replicate the environment in which many receptors function, including cell membrane bound receptors, such as the G-protein coupled receptors, or “GPCRs” as they will be referred to hereafter.

The GPCRs are integral membrane proteins. They constitute the largest family of receptors in the human genome, and are also seen in other mammalian, and non-mammalian species, including primates, canines, insects, and so forth. With respect to drug discovery, it has been estimated that over 50% of therapeutic agents that are on the market or are in development are directed at GPCRs as their target. See Edwards, et al, Trends Pharmacol. Sci. 21:304-308 (2000). Hence, there is considerable interest in developing cell free assays for target molecules in general, receptors in particular, and the GPCRs most particularly.

Neumann, et al, ChemBioChem 3:993-998 (2002), incorporated by reference in its entirety, discusses one form of cell free assay, using the human β2 adrenergic receptor (“β2AR.”) This is a GPCR that mediates the effect of catecholamines such as epinephrine released by the sympathetic nervous system. The paper referred to, supra discusses direct labeling of β2AR, using a chemical coupling reagent to attach a fluorophore to native cysteine residues. Fluorophores coupled to an endogenous cysteine at position 265 in the β2AR sequence show a change in fluorescence intensity in response to an agonist-induced conformational change in the receptor protein. The β2AR receptor was also modified to include a “FLAG” sequence at the N-terminus to facilitate labeling with antibody, and a histidine tag of 6 histidines at the C-terminus. The fluorophore-labeled β2AR is immobilized on an avidin or streptavidin coated surface, using a biotinylated antibody that binds the N-terminal FLAG epitope. Jensen, et al, J. Biol. Chem. 276(12);9279-9290 (2001), incorporated by reference, describes another system of this type, as does Bieri, et al, Nature Biotech. 17:1105 (1999).

While the approach disclosed by Neumann et al, and Jensen, et al, is not without interest, it requires the unique presence of a chemically reactive amino acid residue such as cysteine in a position that is sensitive to conformational changes in the receptor. Alternately, it requires the modification of each receptor sequence to introduce such a reactive amino acid residue in a conformation-sensitive position and to delete similarly reactive residues in other positions. It further requires that these modifications and the fluorophore coupling reaction do not impair the receptor activity and ligand binding properties. Because of these constraints, it would be useful to have an approach available to carry out cell free assays on any receptor where the native amino acid sequences of the receptor are modified as little as possible. It would also be useful to have an assay system available where the reporter molecules are not covalently coupled to the native receptor protein, but rather are reversibly bound using a metal complexing procedure.

The invention, which is set forth in the disclosure which follows, addresses these and other issues, as will be seen by consideration thereof.

### BRIEF DESCRIPTION OF THE FIGURES

FIG. 1 shows a flow diagram for production and analysis of a labeled receptor used in the invention.

FIG. 2 shows the dependence of fluorescence of a perylene derivative on the dielectric constant of the solvent used.

FIG. 3 shows the structure of various perylene derivatives described in the invention, including Ni²⁺ chelated compounds with a chelating groups at one end (bottom right) and at both ends (bottom left), attached to a histidine chain containing protein via Ni²⁺.

FIG. 4 is a flow-chart/schematic showing how to purify complexes of nickel-perylene and receptor, via chromatography.

FIG. 5 presents a second flow chart, showing how to obtain the complexes of the invention in an immunprecipitation binding assay.

FIG. 6 elaborates data presented schematically in FIG. 5, including results of washes and elutions of the desired complexes.

FIG. 7 presents further work set forth in FIG. 5, using a control perylene derivative that lacks a chelating group.

FIG. 8 shows how the poly(His) receptors of the invention, positioned in a lipid bilayer of, e.g., a cell, are solubilized in a detergent micelle solution, to which the Ni²⁺ perylene compounds can be added.

FIG. 9 shows how the invention functions.

FIG. 10 shows a one application of the invention, where a plurality of receptors, are presented on a microarray to determine which receptors are activated by a given test compound.

FIG. 11 presents a further application of the invention, which is a microarray designed to determine if a particular compound or compounds are present in a test sample by determining if receptor complexes sensitive to the compound are activated.

### DETAILED DESCRIPTION OF PREFERRED EMBODIMENTS

In the description of the invention, reference will be made to both general, and specific embodiments of various portions thereof. Reference to specifics are given to explain the invention in greater detail, and/or to provide an example of particularly preferred embodiment. They are not to be taken as limiting the invention in any way.

In the practice of the invention, one requires a supply of receptor molecule of interest. While there are various, non-recombinant ways of obtaining pure, receptor proteins, it is most preferred, for reasons well known to the skilled artisan, to prepare these receptor molecules recombinantly. Such recombinant preparation may take place in a prokaryotic cell system, such as E. coli, or a eukaryotic system, such as Spodoptera frugiperda, or some other cell type that is easily transfected with, a plasmid or viral expression vector.

In order to make the receptor molecules recombinantly, it is preferred to introduce a recombinant construct, e.g., an expression vector, into a host cell, such as those referred to supra.

The expression vectors used in the invention are constructed such that they comprise, at a minimum, a coding region which encodes a protein or a portion of a protein which facilitates movement of the receptor molecule of interest to the extracellular membrane, a coding region which encodes the receptor of interest, and a modification of the receptor, which facilitates binding of a reporter molecule thereto.

In a preferred embodiment of the invention, when E. coli cells are used as the host cells, the protein which facilitates the movement to the extracellular membrane is one which is normally transported to the periplasm of the cell. Maltose binding protein, or “MBP” is an example of one such protein. The construct encoding the MBP is placed 5′ to the construct which encodes the modified receptor. The construct is chosen such that a cleavage site is placed in between the two components, e.g., MBP and the receptor protein, so that at a convenient point in time, the transport protein may be cleaved therefrom. See Tucker, et al, Biochem. J. 317(Pt3):891-9(1996), incorporated by reference, for a description of such an MBP system.

The receptor may be any of the receptors known to the art. The “G-protein coupled receptors” represent one family of great interest. Exemplary of the receptors in this family are the β2 adrenergic receptor, or “β2AR,” serotonin receptors, such as 5HT1A, 5HT2C, and others that are well known to the art. Preferred systems for expression of GPCRs are well known. See Tate, et al, Trends Biotechnol. 14(11):426-430 (1996), incorporated by reference.

The region encoding the receptor, as was noted supra, is modified such that, when translated, the molecule contains a binding site to which the reporter molecule binds effectively. In an especially preferred embodiment of the invention, this is a “poly(His)” sequence, which contains a sequence of histidine molecules, of formula (His)ₙ, where n is a whole number between 3 and 30, preferably 3 and 20, more preferably 5 and 15, and most preferably 5 to 10. These poly His structures are known to bind to transition metal ions, such as Ni²⁺, which in turn chelate to chelating agents which can be attached to reporter molecules. Other metal/chelator systems are known and can be used as is evidenced by, e.g., Hochuli, et al, J. Chromatogr. 411:177-184 (1987), incorporated by reference.

The poly (His) sequence may be attached to the C terminus of the receptor, or may be inserted at any point within the receptor which does not result in serious impairment of the receptor function. For example, in the case of the GPCRs described supra, the poly (His) sequence may be inserted in any of the cytoplasmic loops between the transmembrane domains, or in the cytoplasmic tail following the seventh transmembrane domain. As a further example, the GPCR may be expressed as a fusion to a G-protein, wherein the C-terminus of the receptor is fused to the N-terminus of the G-protein alpha subunit. See Milligan, Methods Enzymol. 343:260-273 (2002) for a discussion of such receptor-G protein fusion systems. In such fusions, the poly(His) sequence may be inserted at the junction between the receptor and the G-protein, or at the C-terminus of the G-protein fusion partner. More than one poly(His) or other binding structure can be added to the molecule. Other points of attachment for the poly(His) sequence are possible and these, as well as methods for their incorporation, will be known to the skilled artisan.

Once the construct, referred to supra, is expressed, it is purified from the cell. Many methods are known for how to accomplish this, and as such will only be discussed briefly herein. As the constructs have been designed to transport the receptor protein of interest to the cell membrane, the purification protocol preferably isolates membrane fractions of the cells. “Proof of principle,” i.e., did the cells express the receptor of interest, can be determined very easily. Many receptors, including the GPCRs referred to supra, are known to contain an N-terminal region which crosses the cell membrane, followed by a plurality of domains which are transmembrane domains, and a C terminal, intracellular region. As this is the milieu in which these receptors function normally, one determines their presence and activity by adding a ligand known to bind to the receptor and then determining if binding occurs.

Once the receptor is known to be present in the membrane extract, it is solubilized by adding a detergent, preferably a non-ionic detergent, such as an alkyl maltoside, or n-dodecyl-β-D maltoside. See Weiss, et al, “MRC Laboratory of Molecular Biology” available through NCBI, to show the use of this detergent, in purifying receptors. Other non-ionic detergents are known to the artisan, and need not be presented here.

The proteins are then separated via, e.g., affinity chromatography or other methods known in the art. The protein portion used to transport the receptor to the cell membrane may be removed at this point, but it need not be.

Once the receptor is isolated, it is necessary to bind the reporter molecule to it. Many examples of systems for linking a reporter molecule to a protein, such as a receptor, are known and need not be reiterated here. Preferably, however, the reporter molecule is linked to the receptor via a metal bridged complex, in which a transition metal ion, such as Ni²⁺, Zn²⁺, or Co²⁺, binds to the chelating group of the reporter molecule at one side and to the polyhistidine chain of the target molecule (e.g., GPCRs) at the other side. Various chelating agents are known, including iminodiacetic acid, or “IDA,” and nitrilotriacetic acid, or “NTA,” which is preferred.

The reporter molecule may be any substance which generates a discemable signal upon interaction of the receptor with a ligand. The signal may be chemiluminescent, calorimetric, radioactive, and is preferably fluorescent. Of particular interest and use in the invention is the class of compounds known as perylenes. These fluorescent molecules possess a symmetric ring structure, including two imide nitrogen atoms at both sides, which permit the attachment of chelating groups to the molecule, which in turn facilitates the binding to Ni²⁺ and the histidine chain.

In the practice of the invention, it has been found that perylenes exhibit exquisite sensitivity when used in the assay of the invention. When these assays are described hereafter, reference will be made to perylenes as the “reporter” or “indicator” molecules, it being understood that other molecules may also be used.

In making the fluorescent tagged complexes, the histidine chain containing receptor, nickel, and chelating agent-perylene complex are combined and incubated at 4° C., overnight to facilitate formation of the complex. Free molecules can be separated from complexes via, e.g., gel filtration, affinity chromatography or other standard separation methods.

In a preferred embodiment, the formation of the complexes takes place in a solution of a non-ionic detergent, such as the detergent described supra, so as to form micelles which contain the complexes.

It was noted, supra, that perylene molecules and their derivatives contain two imide moieties, to which chelating groups can be joined. In addition to the chelating groups, various hydrophobic or hydrophilic groups may be attached at these positions. The groups attached to each nitrogen can be the same or different. It is well known that detergent micelles and lipid bilayers present a hydrophilic exterior and a hydrophobic interior portion. Hence, a hydrophobic moiety, if used, can help to direct the perylene reporter to the interior of the micelle or lipid bilayer, and to the hydrophobic portions of the receptor. Hydrophilic or charged moieties instead direct the perylene reporter to the exterior of the micelle or lipid bilayer, and the solvent-exposed, hydrophilic portions of the receptor.

As noted, supra, in practice the complexes of the invention are used, in a cell free system such as a non-ionic detergent micelle, although this is not required. The complexes may also be incorporated into an artificial lipid bilayer. A substance of interest is then admixed with the indicator system, and changes in the fluorescence, such as fluorescence intensity, can be measured and compared to a control, to determine if there has been interaction, and if so, the extent thereof. Such a comparative assay can be used to determine agonistic or antagonistic properties of a molecule, such as by comparing signal obtained with the substance of interest to a value obtained using a known ligand.

The molecular complexes of the invention and the micelles which incorporate them may be used to produce apparatuses, where these materials are affixed to a solid phase, such as glass, plastic, or a microchip. Such apparatuses can contain multiple copies of one type of molecular complex or a mixture of various types of molecular complexes. Which type to use will depend upon the type of assay under construction.

Attachment to the solid phase may be accomplished in any of the ways known to the art. As was pointed out, supra, one embodiment of the invention includes maltose binding protein at the N-terminus. One can attach the molecular complexes to the solid phase, via an anti-MBP antibody, possibly via the intermediary of a biotin-(strept)avidin system. Other types of epitope tag, including but not being limited to MYC, HA, or FLAG could also be used, again possibly through the intermediary of a biotin-(strept)avidin system.

More details and the practice of the invention will be seen via a review of the examples which follow.

### DETAILED DESCRIPTION OF THE PREFERRED EMBODIMENTS

### EXAMPLE 1

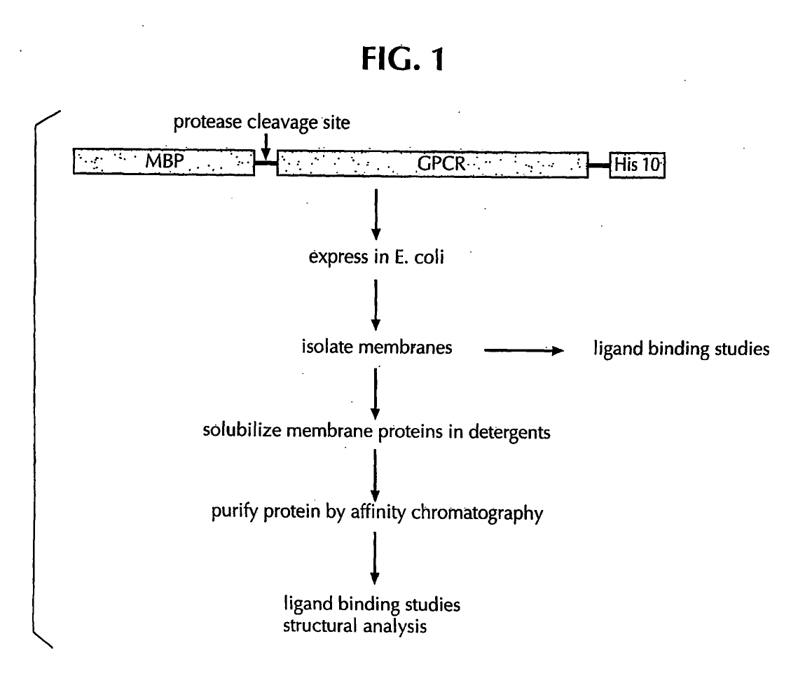

This example describes the manufacture of His labeled receptors. It is depicted, schematically, in FIG. 1.

For the labeling of receptors with a His chain at the C-terminus, an oligonucleotide sequence, i.e., SEQ ID NO: 1 was prepared, which encoded a string of 10 His residues. The sequence, ctagtgccgc gcggcagcca ccaccaccac caccaccacc accaccactag, encodes Leu Val Pro Arg Gly Ser (His)10—stop. The sequence was designed to provide a cleavage site for thrombin protease, so that the tag could be removed. Given the degeneracy of the genetic code, it is to be understood that any oligonucleotide molecule capable of encoding a His chain in accordance with the invention could be substituted for SEQ ID NO: 1.

The sequence was generated at the C-terminus of cDNA encoding each of (i) rat serotonin receptor 5HT2C, (ii) human β2AR, and (iii) human serotonin receptor 5HT1A, via PCR using standard methods. The nucleotide sequences for each of these is attached hereto, and may also be found via GenBank, using Accession Numbers NM-012765; NM000024, and AB041403, respectively.

For the labeling of receptors with a His chain in the intracellular loop between transmembrane domains 5 and 6, or at the junction between a receptor and a fused G protein alpha subunit, an oligonucleotide sequence, i.e., SEQ ID NO: 2 was prepared, which encoded a string of 6 His residues, preceded by the dipeptide Arg Ser, and followed by the dipeptide Gly Ser. The sequence, agatcccatc atcaccatca ccacggatct encodes Arg Ser (His)6 Gly Ser. Given the degeneracy of the genetic code, it is to be understood that any oligonucleotide molecule capable of encoding a His chain in accordance with the invention could be substituted for SEQ ID NO: 2.

This sequence was generated at three positions in the intracellular loop between transmembrane domains 5 and 6 in the rat serotonin receptor 5HT2C via PCR using standard methods: (i) replacing residues 244-245 (Arg Gln), (ii) replacing residues 299-300 (Arg Gly), (iii) replacing residues 306-307 (Asn Asn). PCR was also used to generate this sequence at the junction of a translational fusion between the rat serotonin receptor 5HT2C and the human G protein alpha subunit G-alpha-q (GenBank Accession Number AF493896) wherein the 3′ end of the sequence encoding the 5HT2C receptor was fused to the sequence encoding Arg Ser (His)6 Gly Ser, which in turn was fused to the N-terminal Met codon of the gene encoding G-alpha-q.

For the labeling of receptor-G alpha fusion proteins with a His chain at the C-terminus, an oligonucleotide sequence, i.e., SEQ ID NO: 3 was prepared, which encoded a string of 6 His residues. The sequence, catcatcacc atcaccacta a encodes (His)6—stop. Given the degeneracy of the genetic code, it is to be understood that any oligonucleotide molecule capable of encoding a His chain in accordance with the invention could be substituted for SEQ ID NO: 3.

This sequence was inserted at the C-terminus of a translational fusion between the rat serotonin receptor 5HT2C and the human G protein alpha subunit G-alpha-q (GenBank Accession Number AFF493896) wherein the 3′ end of the sequence encoding the 5HT2C receptor was fused to a sequence, i.e., SEQ ID NO: 4 encoding Gly Ser (Gly)3 Trp (Gly)3 Ser, which in turn was fused to the N-terminal met codon of the gene encoding G-alpha-q. SEQ ID NO: 4 is ggatccggag gtggatgggg aggtggatcc.

The resulting His tagged cDNA molecules were subcloned into a commercially available vector, i.e., PMAL-p2X, which already contains a coding region for maltose binding protein, in operable linkage, and then transformed into commercially available, BL21 Codon Plus cells, via standard methodologies, and were then plated onto LB plates containing 100 μg/ml ampicilin and 50 μg/ml chloramphenicol, again using standard methods.

A single colony was selected, and used to inoculate LB containing 100 μg/ml ampicillin, and 50 μg/ml chloroamphenicol. The culture was grown overnight, at 37° C., to saturation. The culture was then diluted, 1:100, in 2XYT containing 50 μg/ml ampicillin; and 25 μg/ml chloramphenicol. The diluted culture was then grown to an OD₆₀₀ of approximately 0.5, at 37° C.

Temperature was reduced to 18° C., and IPTG was added to a 0.1 mM final concentration, and then cultures were grown for about 18 hours. Cells were harvested, via centrifugation, and stored at −80° C.

When needed, spheroblasts were prepared by resuspending cell pellets obtained from a 500 ml culture, in 60 mls of ice cold Tris (0.1M, pH8.0). An equal volume of ice-cold 0.1M Tris pH8.0/0.5M sucrose was added, and the cell suspension was mixed gently. EDTA was added, to a final concentration of 0.5 mM, as was lysozyme (0.05 mg/ml, final concentration), and DNAase I (10 ug/ml, final). The cell solution was incubated with rocking, for one hour at 4° C. Spheroblasts were then pelleted via centrifugation at 18,500× g for one hour, at 4° C.

The resulting spheroblast pellet was resuspended in 70 ml of ice cold Tris (pH 7.4, 20 mM) 20 mM NaCl & homogenized in a glass/glass homogenizer, using a tight fitting pestle.

The membranes were recovered via centrifugation at 100,000× g for 2 hours, at 4° C. The membrane pellets were then resusupended in 10 mM of ice cold, 20 mM Tris (pH 7.4)/20 mM NaCl, and homogenized as above.

Membrane proteins were then solubilized, via addition of equal volumes of 2% (w/v) n-dodecyl-β-D-maltoside/0.4% cholesteryl hemisuccinate. The solution was allowed to rock, overnight at 4° C., and remaining insoluble materials were pelleted via centrifugation at 18,500× g for one hour, and removed.

Tris (pH 7.4) was added to the membrane proteins, to a final concentration of 50 mM, NaCl was added to a final concentration of 300 mM, and inidazole was added to a final concentration of 25 mM (for receptors tagged with His10 at the C-terminus) or 10 mM (for receptors tagged with His6 in the intracellular loop or for His6 tagged receptor-G protein fusions). The solution was incubated with rocking at 4° C. for one hour, after which insoluble materials were removed via centrifugation, at 18,500× g for one hour, at 4° C. One ml of a 50% slurry of commercially available Ni-NTA superflow beads were added to the solubilized membranes, and were incubated, with rocking overnight, at 4° C.

The slurry was then loaded into a disposable column for chromatography, washed with 25 bed volumes of wash buffer 1 (50 mM Tris, pH 7.4, 300 mM NaCl, 0.2% maltoside/0.04% CHS), containing either 50 mM imidazole (for receptors tagged with His10 at the C-terminus) or 10 mM imidazole (for receptors tagged with His6 in the intracellular loop or for His6 tagged receptor-G protein fusions), followed by a wash with 25 bed volumes of wash buffer 2 (50 mM Tris, pH 7.4, 300 mM NaCl, 0.05% maltoside/0.01% CHS), containing imidazole at the same concentration. His tagged receptor protein was then eluted in elution buffer (50 mM Tris, pH 7.4, 300 mM NaCl, 1M imidazole, 0.05% maltoside/0.01% CHS). Protein containing fractions were pooled, dialyzed against 20 mM Tris, pH 7.4, 150 mM NaCl, 0.05% maltoside, 0.01% CHS to remove imidazole and stored at 4° C.

Western analysis was performed, using a commercially available, anti-MBP antibody, and the results, indicated that the preparation and isolation of the MBP-receptor-poly His complexes was successful. Radiolabeled ligand binding studies using commercially available ³H-LSD (for the serotonin receptor 5HT2C) or ³H-dihydoralprenolol (for the human β2AR) were performed using spheroplast prepared from bacteria expressing the corresponding tagged receptors and the results demonstrated the production of functionally active receptor capable of high affinity binding to an appropriate ligand.

### EXAMPLE 2

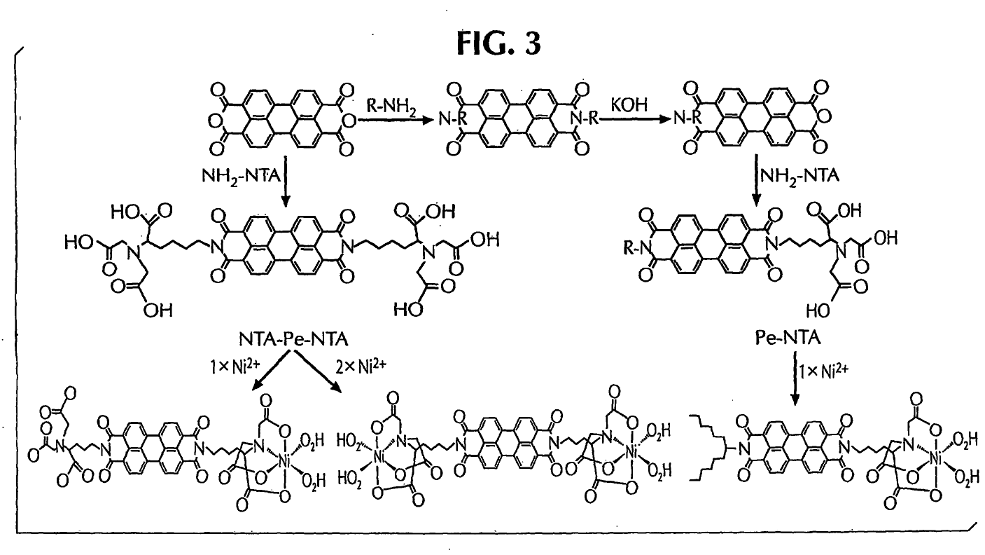

This example, and the example which follows, are exemplary of the synthesis of perylene derivatives which are useful in the practice of the invention. FIG. 3 summarizes the syntheses, with “Pe-NTA” corresponding to the compound described in this example, and “NTA-Pe-NTA” to the compound of example 3, which follows. “Pe-NTA” is an abbreviation for N-(1-hexylheptyl)-N′-(5-(1-carboxyl-1-(N-dicarboxymethyl)pentyl)perylene-3,4,9,10-tetracarboxylic-diimide.

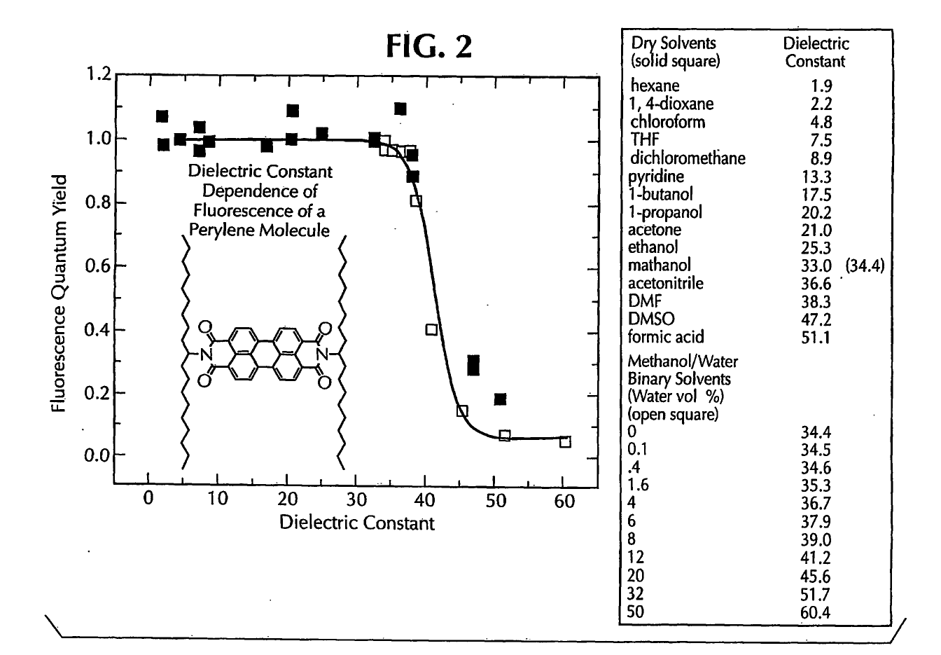

Perylenes are the fluorescent compounds that are preferred in the invention, because the fluorescence quantum yield of this class of compounds such as N,N′-dinonyldecyl-perylene-3,4,9,10-tetracarboxylic-diimide is dependent upon solvent dielectric constants, or “ε”. The range at which they are most sensitive is ε35-45, which is the polarity range of protein/lipid/water interfaces. See FIG. 2. As this interface is present in assays in accordance with the invention, this class of molecules is suitable for membrane/protein interface probing.

In order to synthesize the Pe-NTA, known methods were used. In brief, 17 mg (0.013 mmol) of N-(1-hexylheptyl)perylene-3,9,10-tetracarboxylic-3,4-anhydride 9,10-imide which was synthesized from perylene-3,4,9,10-tetracarboxylic-dianhydride using known methods, was combined with 75 mg (0.29 mmol) N_{α}-N_{β}-bis (carboxymethyl)-L-lysine hydrate, in 750 mg imidazole. The reaction mixture was stirred at 100° C. under argon, for 2 hours. It was cooled to room temperature, and then 2M HCl was added, to dissolve the red solid which had formed. Excess HCl was added, to bring the reaction pH to about 2, at which point the reaction product precipitated out of solution, forming a suspension.

The suspension was vacuum filtered through a 0.45 μm membrane filter, and the red solid which was collected was washed, thoroughly, with distilled water, until the pH of the washing solution was neutral. Solid was dried, in a vacuum, at 100° C. for 3 hours.

Chemical structure and purity were determined via NMR, in 1:1 CDCl₃:CD₃COOD binary solvents. The purity was about 100%.

The structural data for the compound are as follows. H-NMR: δ=0.86 (t, 6H, 2CH₃), 1.3 (m, 18H, 9CH₂), 1.86 (m, 2H, α-CH₂), 1.9 (m, 2H, CH₂), 2.3 (m, 2H, α-CH₂), 3.56 (t,1H, CH), 3.74 (s, 4H, 2CH₂), 4.26 (t, 2H, CH₂), 5.25 (m, H, CH), 8.7-8.8 (broad, 8H, perylene).

### EXAMPLE 3

In this example, the synthesis of “NTA-Pe-NTA,” or N, N′-di (5-(1-carboxyl-1-(N-dicarboxyl methyl)pentyl) perylene-3,4,9,10-tetracarboxylic-diimide is described.

A 21 mg (0.054 mmol) sample of perylene-3,4,9,10-tetracarboxylic-dianhydride was combined with 142 mg (0.542 mmol) of the L-lysine derivative of example 2, together with 1 g of imidazole, and heated at 105° C. for 3 hours, under argon. The mixture was cooled to room temperature, and 2M HCl was added, again to dissolve the red solid. Similarly to example 2, excess HCl was added, to a pH of about 2, to precipitate out reaction product. The suspension was filtered, as described, supra. The product was vacuum dried at 95° C. for 2 hours. NMR was carried out, in 4:6 CD₃ COOD: DMSO-d₆ binary solvents. Purity was about 100%.

The structural data are as follows: ¹H-NMR: δ=1.35-1.8 (m, 12H, 6CH₂), 3.35-3.48(m, 2H, 2CH), 3.4-3.64 (m, 8H, 4CH₂), 3.95 (broad, 4H, 2CH₂), 7.95-8.25 (broad, 8H, perylene).

### EXAMPLE 4

This example describes the addition of nickel to the alkyl-Pe-NTA compound of example 2, to form “alkyl-Pe-NTA-Ni,” “compound 1” hereafter, or N-(1-hexylheptyl)-N′-(nickel (II)-5-(1-carboxyl-1-(N-dicarboxyl methyl)pentyl))perylene-3,4,9,10-tetracarboxylic-diimide. A 2 mg (0.0038 mmol) sample of the product described in example 2 was dissolved in 2 mL of 1:1 chloroform/methanol mixed solvent. NiCl₂·6H₂O solution (4 mg; 0.017 mmol) was added, followed by addition of 0.5M NaCl aqueous solution, dropwise with stirring. Precipitate formed at the chloroform/water interface.

Chloroform was removed under a vacuum, and the aqueous precipitate was filtered, as described supra, and washed with 0.5M NaCl and water.

The resulting compound 1 was dissolved in 2% -(w/v) n-dodecyl-β-D-maltoside/0.4% cholesteryl hemisuccinate (CHS) via sonication.

### EXAMPLE 5

Similarly to example 4, nickel was added to the compound that is the product of example 3. The compound is referred to as “NTA-Pe-NTA-Ni,” or “compound 2” herein, or more completely N-(5-(1-carboxy-1-(N-dicarboxy methyl) pentyl))-N′-(nickel (II)-5-(1-carboxyl-1-(N-dicarboxy methyl)pentyl)) perylene-3,4,9,10-tetracarboxylic-diimide.

A 0.002 mmol sample (1.89 mg), of the product of example 3 was dissolved, via sonication, in 4 mL of 20 mM Tris (pH 7.4), 150 mM NaCl, 1% maltoside, 0.2% CHS. Another 4 mL of 20 mM Tris (pH 7.4), 150 mM NaCl, 1% maltoside, 0.2% CHS containing 0.5% mM Ni²⁺ ions was added to yield a final concentration of the 1:1 nickel chelated complex, i.e., the NTA-Pe-NTA-Ni, of 0.25 mM.

### EXAMPLE 6

The product of example 3 was then treated to add two nickel ions, to form “Ni-NTA-Pe-NTA-Ni,” or N, N′-di(nickel II)-5-(1-carboxyl-1-(N-dicarboxy methyl)pentyl))perylene-3,4,9,10-tetracarboxylic-diimide. To do this, the protocol of example 5 was followed, except the amount of Ni²⁺ used was doubled, to 1.0 mM. The final concentration was 0.25 mM.

### EXAMPLE 7

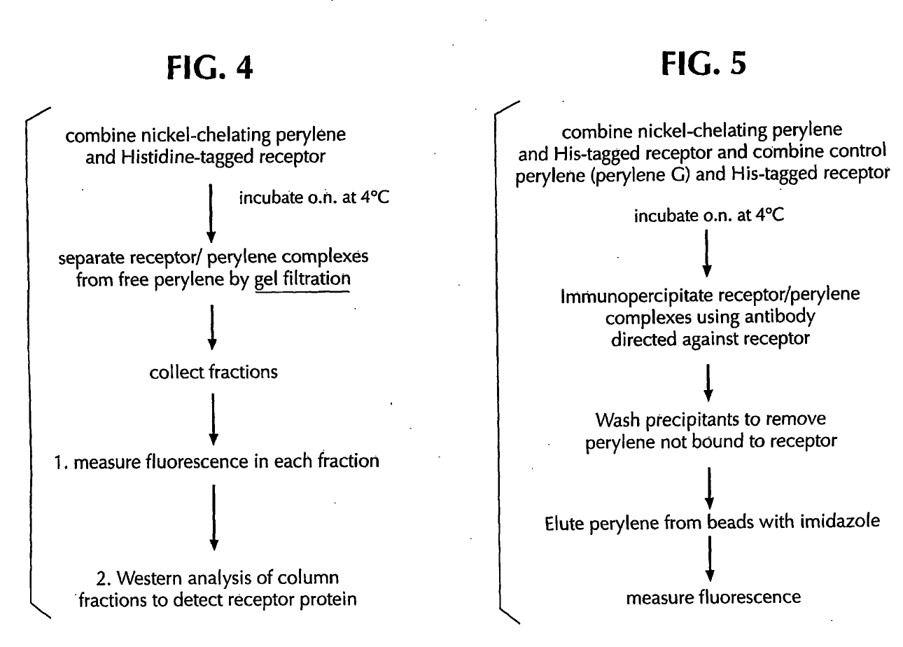

This example describes the chelation of the compound of example 4 to any of the receptor molecules described in example 1 and purification of the receptor-perylene complexes by gel filtration chromatography. Schematically, the chelation and purification are shown in FIG. 4.

The receptor was affinity purified, following standard methods, and 1.1 μM of the molecule was combined with 2.5 μM of compound 1, in a total volume of 0.5 ml. The materials were incubated on a rocking platform, overnight, at 4° C. The mixture was then loaded onto a 20 ml column of Superose 6 Prep gel filtration media, and washed with running buffer (20 mM Tris, pH 7.4, 150 mM NaCl, 0.05% maltoside/0.01% CHS), to remove any unbound perylene. A total of 60 separate, 0.5 ml fractions were collected. A portion of each fraction was loaded into 60 wells of a 96 well plate, and fluorescence was measured at an excitation of 485 nm, and emission of 535 nm. Since the fluorescence quantum yield of perylene bound to receptor differs from that of unbound perylene, fluorescence was also measured after dissociating receptor-perylene complexes by adding imidazole to a final concentration of 0.5M to each of the fractions. A portion of each column fraction was loaded onto denaturing polyacrylamide gel, separated by electrophoreses, transferred to PVDF membranes, and receptor protein containing fractions were identified, using anti-MBP antibodies. Results indicated that roughly one-third of the total perylene was bound to receptor protein and these receptor-perylene complexes were separated from the remaining free perylene.

### EXAMPLE 8

In these experiments, the affinity purified receptor (0.2 μM), and either compound 1, or control glycine-perylene (0.4 μM), were combined, in a total volume of 0.1 ml, and incubated on a rocking platform overnight, at 4° C. Further controls were provided, by taking samples of the two perylene molecules and treating them in the absence of protein.

All samples were incubated overnight with 2.5 μl of anti-MBP antisera, and antibody-protein-perylene complexes were precipitated with 100 μl of protein A, conjugated to Sepharose beads, followed by 3, ten minute washes with detergent solution (20 mM Tris pH 7.4, 150 mM NaCl, 0.05% maltoside/0.01% CHS). All washes were retained for fluorescence measurements.

Perylene molecules were eluted from the bead bound proteins using 0.5M imidazole. Fluorescence of all samples and washes was measured, using a spectrofluorometer, excitation at 480 nm, and emission scan at 500-650 nm. Peak fluorescence was detected at 533-535 nm.

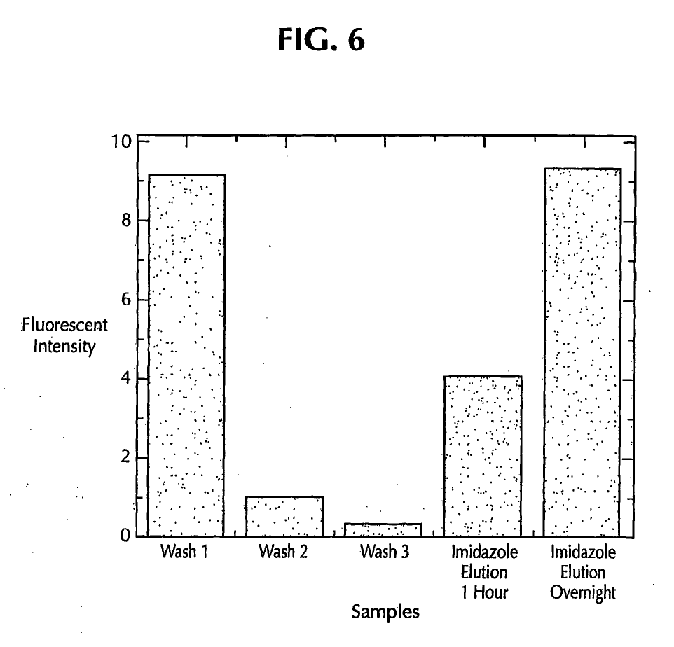

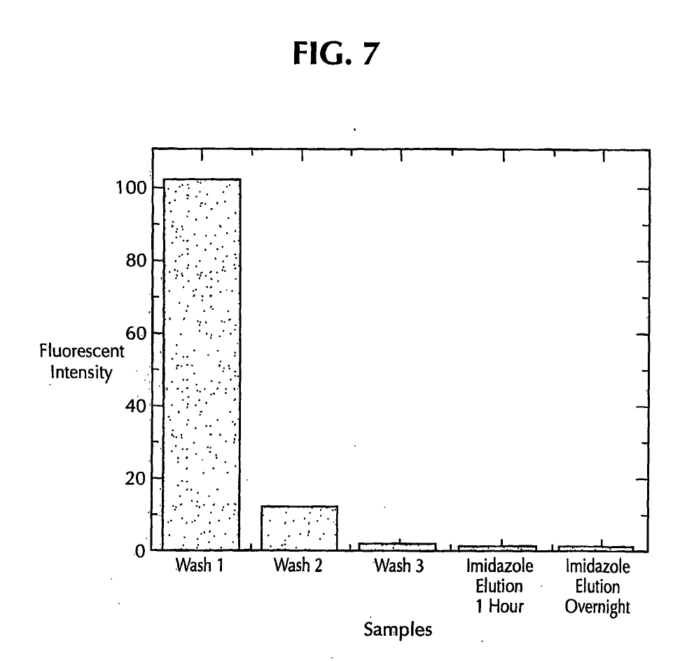

FIGS. 5-7 summarize these data for the binding of compound 1 to the serotonin receptor 5HT2C with a C-terminal His tag described in example 1. Similar results were obtained in experiments designed to test the binding of compound 1 to the other tagged receptors described in example 1. FIG. 5 shows the schematics of the purification system, while FIG. 6 & 7 show, respectively, the fluorescence values obtained from the receptor complexes, and the controls. Note that the key difference is the difference in values following overnight elution, indicating that the chelated compounds bound to the receptor and required elution, while the control compounds did not.

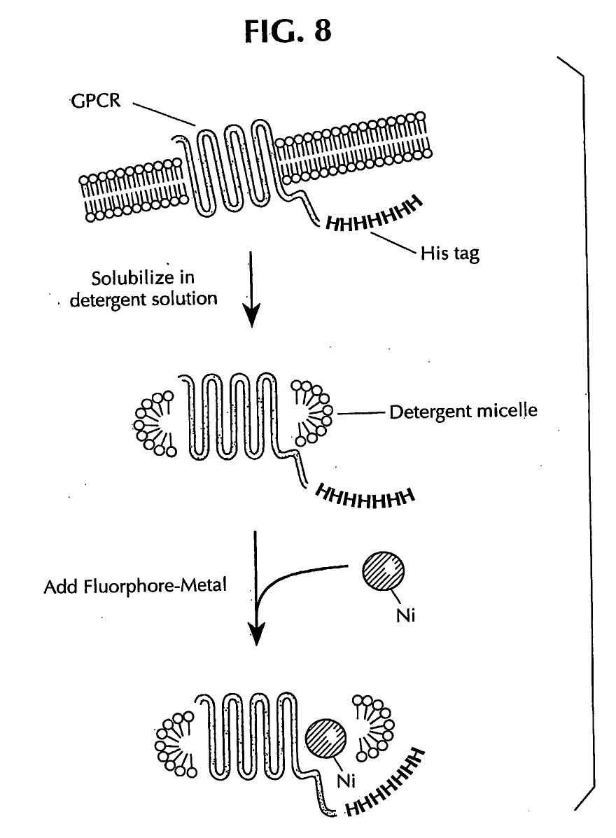

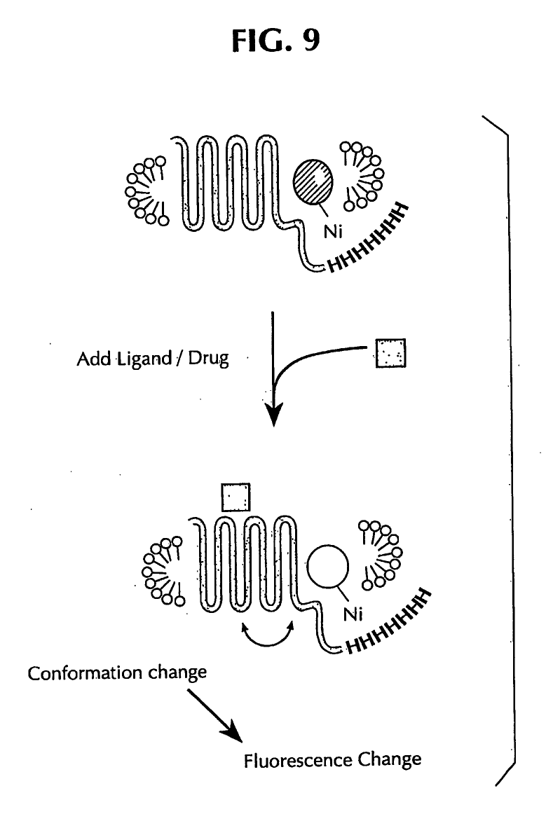

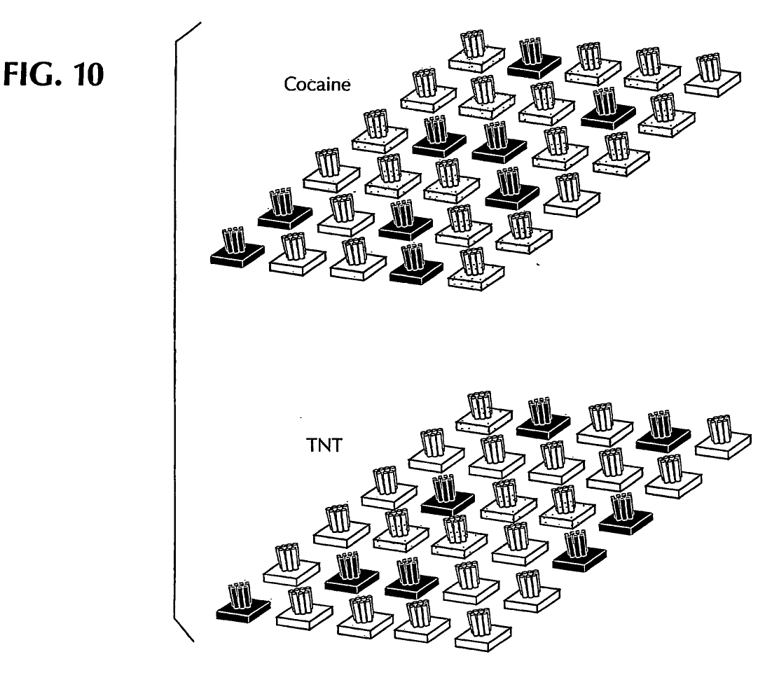

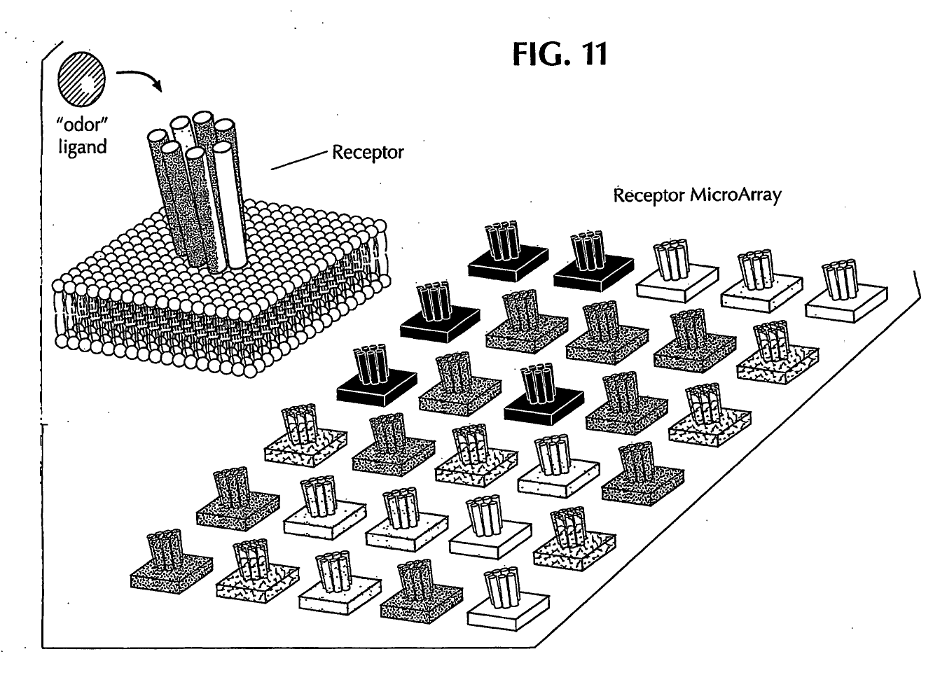

FIGS. 8 & 9 show how the micelles and lipid bilayer constructs of the invention can be obtained, and then used, respectively. See, e.g., Sachmann, Science 271:43-48 (1996), incorporated by reference, for additional information on these systems. The complexes can then be attached to solid phases, such as microchip arrays, as depicted in FIGS. 10 & 11. In FIG. 10, a solid phase, such as a microarray, is presented, with a plurality of receptors bound thereto. Such a microarray may be used for determining which receptor or receptors are activated by a particular compound or compounds. These assays could be used for screening the activity of a given compound on a number of known receptors to determine, for example, the potential for undesired side-effects. Similarly, such a microarray assay can be used to identify receptors for a compound not known previously. A second embodiment of the invention can be seen in FIG. 11, where one can determine whether a particular compound or compounds are present in a sample, by screening with an array which includes one or more receptors that are activated by the compound(s).

Other aspects of the invention will be clear to the skilled artisan and need not be reiterated here.

We claim:

1. A substantially pure molecular complex which comprises:
(i) a receptor protein with an amino acid sequence, having an N terminus and a C terminus, said amino acid sequence comprising a chain of n contiguous histidine residues, where n is a whole number that is 5 or more, and
(ii) a reporter molecule linked to said receptor molecule via said (His)ₙ chain, wherein said reporter molecule generates a detectable signal upon interaction of said receptor and a ligand which interacts with said receptor.

2. The substantially pure molecular complex of claim 1, wherein said reporter molecule is chelated to said (His)ₙ chain.

3. The substantially pure molecular complex of claim 2, wherein said reporter molecule is chelated to said (His)ₙ chain via a chelating agent which comprises a metal ion.

4. The substantially pure molecular complex of claim 3, wherein said metal ion is polyvalent.

5. The substantially pure molecular complex of claim 4, wherein said polyvalent metal ion is Ni²⁺, Co²⁺, or Zn²⁺.

6. The substantially pure molecular complex of claim 5, wherein said polyvalent metal is Ni²⁺.

7. The substantially pure molecular complex of claim 1, wherein said reporter molecule is fluorescent.

8. The substantially pure molecular complex of claim 7, wherein said fluorescent molecule is perylene or a perylene derivative.

9. The substantially pure molecular complex of claim 1, wherein said receptor is a GPCR.

10. The substantially pure molecular complex of claim 1, further comprising a protein or portion of a protein positioned at the N-terminus of said receptor which facilitates transport of said molecular complex to the extracellular membrane of a cell in which it is produced.

11. The substantially pure molecular complex of claim 10, wherein said protein is maltose binding protein.

12. The substantially pure molecular complex of claim 1, wherein said (His)ₙ chain is positioned at the C terminus of said receptor.

13. The substantially pure molecular complex of claim 1, wherein receptor comprises at least 6 transmembrane domains, and has an intracellular loop between the 5^{th} and 6^{th} transmembrane domains, and said (His)ₙ chain is positioned in the intracellular loop between transmembrane 5 and 6 of said receptor.

14. The substantially pure molecular complex of claim 1, wherein said receptor is fused to a G-protein alpha subunit to form a fusion protein and said (His)ₙ is positioned between said receptor and said G protein alpha subunit or at the C-terminus of said fusion protein

15. The substantially pure molecular complex of claim 1, further comprising at least one additional hydrophilic moiety positioned on said indicator molecule.

16. The substantially pure molecular complex of claim 1, further comprising at least one hydrophobic moiety positioned on said indicator molecule.

17. A micelle which comprises the substantially pure molecular complex of claim 1, and a non-ionic detergent.

18. The micelle of claim 1, wherein said non-ionic detergent is n-dodecyl-β-D-maltoside.

19. A method for determining if a substance interacts with a receptor molecule, comprising contacting said substance to the substantially pure molecular complex of claim 1, and determining said detectable signal as an indication of interaction between said substance and said receptor.

20. A method for determining if a substance interacts with a receptor molecule, comprising contacting said substance with the micelle of claim 17, and determining said detectable signal as an indication of interaction between said substance and said receptor.

21. A method for determining if a substance of interest is an antagonist of a receptor comprising contacting said substance to either the substantially pure molecular complex of claim 1 or the micelle of claim 17 in the presence of a known ligand for the receptor, detecting said signal, and comparing said signal to a signal obtained with said ligand alone, wherein a difference in said signals indicates that said substance is a possible antagonist for said receptor.

22. Apparatus useful in determining if a substance binds to a receptor, comprising the substantially pure molecular complex of claim 1 or the micelle of claim 17, affixed to a solid phase.

23. The apparatus of claim 22, comprising a plurality of said substantially pure molecular complexes of micelles.

24. The apparatus of claim 23, wherein said plurality of substantially pure molecular complexes or micelles are the same.

25. The apparatus of claim 23, wherein said plurality of substantially pure molecular complexes or micelles are different.

26. The apparatus of claim 22, wherein said solid phase is a glass slide, a plastic material, or a microchip.

27. A composition comprising a lipid bilayer having inserted therein the substantially pure molecular complex of claim 1.

28. A compound comprising a lipid bilayer having inserted therein a receptor protein with an amino acid sequence having an N-terminus and a C terminus, said amino acid sequence comprising a chain of n contiguous histidine residues, where n is a whole number of 5 or more.
